(19) United States
(12) Patent Application Publication
(10) Pub. No.: US 2024/0060978 A1
(43) Pub. Date: Feb. 22, 2024

(54) ISOELECTRIC FOCUSING-BASED ENRICHMENT OF CHARGE VARIANT SPECIES

(71) Applicant: Regeneron Pharmaceuticals, Inc., Tarrytown, NY (US)

(72) Inventors: Michael Rosconi, New City, NY (US); Ramya Rao, Chappaqua, NY (US); Jennifer Nguyen, Pleasantville, NY (US)

(21) Appl. No.: 18/221,934

(22) Filed: Jul. 14, 2023

Related U.S. Application Data
(60) Provisional application No. 63/399,540, filed on Aug. 19, 2022.

(51) Int. Cl. G01N 33/569; G01N 1/34; G01N 27/447; B01D 15/36

(52) U.S. Cl. CPC G01N 33/56983; G01N 1/34; G01N 27/44791; G01N 27/44795; B01D 15/361; G01N 2333/165; G01N 2469/10

(57) ABSTRACT

The present invention generally pertains to methods of enriching charge variants of a protein of interest. In particular, the present invention pertains to the use of normal polarity or reverse polarity imaged capillary isoelectric focusing and iterative fractionation to enrich charge variant species of interest.

### CROSS-REFERENCE TO RELATED APPLICATIONS

This application claims priority to and the benefit of U.S. Provisional Patent Application No. 63/399,540, filed Aug. 19, 2022 which is herein incorporated by reference

### FIELD

This application relates to methods for enrichment of charge variants of therapeutic proteins.

### BACKGROUND

Biophysical properties, including domain-specific variants, of therapeutic peptides and proteins can affect their safety, efficacy and shelf-life. For example, the presence of different charge variants may alter protein solubility, binding, and stability.

Therapeutic peptides or proteins, such as antibodies, may acquire different variants and become heterogeneous due to various post-translation modifications (PTMs), protein degradation, enzymatic modifications, and chemical modifications. These alterations to biophysical properties may occur at almost any point during and after peptide and protein production. Because these alterations to biophysical characteristics may affect the safety, efficacy, and shelf-life of therapeutic peptides and proteins, it is important to identify different variants for particular therapeutic peptides or proteins, and furthermore to interrogate the modifications responsible for charge variants.

Imaged capillary isoelectric focusing (icIEF) is an industry standard tool for analyzing charge heterogeneity and has been routinely used as a product release method. Variant enrichment is also a valuable tool for understanding the effects of charge sub-populations on drug efficacy, and can help define product specification limits. However, most current enrichment methods rely on ion-exchange chromatography, and the resulting fractions may not always correlate with release icIEF peak profiles.

Therefore, it will be appreciated that a need exists for methods to specifically and sensitively enrich charge variants of a therapeutic protein in a manner consistent with icIEF charge heterogeneity analysis.

### SUMMARY

A method has been developed for enrichment of charge variants of a protein of interest. In an exemplary embodiment, a sample including a protein of interest is subjected to capillary isoelectric focusing (cIEF) analysis. The cIEF analysis may be conducted in normal polarity or reverse polarity. A UV trace of the protein sample is generated, which includes UV peaks corresponding to charge variants. The focused sample is then mobilized out of the separation column, and fractions of the sample are collected. A charge variant of interest may be enriched in multiple fractions, in which case multiple fractions may be combined to produce a sample enriched for the charge variant of interest. The cIEF analysis and fraction collection may be repeated at least once, and fractions including the charge variant of interest across all analyses may be combined to produce a sample enriched for the charge variant of interest. The sample may be a partially enriched charge variant sample, for example a fraction collected from an ion exchange chromatography analysis.

These, and other, aspects of the invention will be better appreciated and understood when considered in conjunction with the following description and accompanying drawings. The following description, while indicating various embodiments and numerous specific details thereof, is given by way of illustration and not of limitation. Many substitutions, modifications, additions, or rearrangements may be made within the scope of the invention.

### BRIEF DESCRIPTION OF THE DRAWINGS

FIG. 1 shows electropherograms of two lots of mAb1 formulated drug substance (FDS), according to an exemplary embodiment.

FIG. 2A shows electropherograms of two lots of mAb1 FDS using two broad range ampholyte products, according to an exemplary embodiment.

FIG. 2B shows electropherograms of two lots of mAb1 FDS using two narrow range ampholyte products, according to an exemplary embodiment.

FIG. 3 shows overlaid electropherograms of back-to-back icIEF injections of mAb1 DS, according to an exemplary embodiment.

FIG. 4 shows electropherograms of mAb1 FDS fractions, according to an exemplary embodiment.

FIG. 5 shows electropherograms of mAb1 unfractionated FDS compared to a mAb1 fraction enriched for a charge variant of interest, according to an exemplary embodiment.

FIG. 6A illustrates a target-binding assay for mAb1, according to an exemplary embodiment.

FIG. 6B shows a signal of mAb1 binding to a target molecule over time for unfractionated FDS compared to a fraction enriched for a charge variant of interest, with varying concentrations of target molecule, according to an exemplary embodiment.

FIG. 7A shows an electropherogram of mAb2 DS, according to an exemplary embodiment.

FIG. 7B shows separation of mAb2 DS fractions using icIEF, according to an exemplary embodiment.

FIG. 7C shows electropherograms of mAb2 DS fractions, according to an exemplary embodiment.

FIG. 8A illustrates the order of mobilization of charged variant species using normal polarity icIEF, according to an exemplary embodiment.

FIG. 8B illustrates the order of mobilization of charged variant species using reverse polarity icIEF, according to an exemplary embodiment.

FIG. 9A illustrates a two-step ion exchange chromatography enrichment process for mAb2 DS, according to an exemplary embodiment.

FIG. 9B shows electropherograms of mAb2 DS fractions, according to an exemplary embodiment.

FIG. 10A shows separation of mAb2 DS fractions using normal polarity icIEF, according to an exemplary embodiment.

FIG. 10B shows electropherograms of fractions collected from mAb2 DS using normal polarity icIEF, according to an exemplary embodiment.

FIG. 10C shows separation of mAb2 DS fractions using reverse polarity icIEF, according to an exemplary embodiment.

FIG. 10D shows electropherograms of fractions collected from mAb2 DS using reverse polarity icIEF, according to an exemplary embodiment.

FIG. 11A illustrates charge variants of mAb3 DS separated using strong cation exchange chromatography, according to an exemplary embodiment.

FIG. 11B shows an electropherogram of a mAb3 charge variant fraction, according to an exemplary embodiment.

FIG. 12A shows separation of proxy pI markers using reverse polarity icIEF, according to an exemplary embodiment.

FIG. 12B shows electropherograms of fractions collected from proxy pI markers using reverse polarity icIEF, according to an exemplary embodiment.

FIG. 13A shows overlays of separations of a mAb3 charge variant fraction using reverse polarity icIEF, according to an exemplary embodiment.

FIG. 13B shows electropherograms of fractions collected from a mAb3 charge variant fraction using reverse polarity icIEF, according to an exemplary embodiment.

### DETAILED DESCRIPTION

Therapeutic antibodies produced in mammalian cells, such as monoclonal antibodies (mAbs), are heterogeneous as a result of post-translational modifications (PTMs), enzymatic modifications and chemical modifications, which contribute to size and charge variants. These modifications may include, for example, glycosylation, deglycosylation, amidation, deamidation, oxidation, glycation, terminal cyclization, C-terminal lysine variation, C-terminal arginine variation, N-terminal pyroglutamate variation, C-terminal glycine amidation, C-terminal proline amidation, succinimide formation, sialylation, or desialylation. In addition, aggregation, degradation, denaturation, fragmentation, or isomerization of protein products can also introduced charge heterogeneity. Table 1 shows exemplary protein modifications and their impacts on changing the electric charges of peptides or proteins.

TABLE 1
Exemplary protein modifications that may cause charge variants

| | | Species |
|---|---|---|
| Protein modifications | Effect | formed |
| Sialylation | COOH addition | Acidic |
| Deamidation | COOH formation | Acidic |
| C-terminal lysine cleavage | Loss of NH2 | Acidic |
| Adduct formation | COOH formation or loss of | Acidic |
| | NH2 | |
| Succinimide formation | Loss of COOH | Basic |
| Methionine, cysteine, lysine, | Conformational change | Basic |
| histidine, tryptophan oxidation | | |
| Asialylation (terminal galactose) | Loss of COOH | Basic |
| C-terminal lysine and glycine | NH2 formation or loss of | Basic |
| amidation | COOH | |

During the manufacture of a therapeutic peptide or protein, such as a monoclonal antibody, charge heterogeneity is potentially introduced as a result of protein degradation and/or the presence of PTMs. Characterization of charge variant forms of a protein within the manufactured drug substance is required to fully understand the correlation between properties of the protein, such as potency, and the physical and chemical changes associated with the charge variants.

Several methods exist that allow for separation of protein charge variants, including ion exchange chromatography and isoelectric focusing (IEF). IEF has become a more common approach because of its capacity for high-resolution separation of sample components based on pI, and its ability to account for both surface-exposed and internal amino acids with no loss of resolution due to hydrophobic interactions. IEF, particularly imaged capillary IEF (icIEF), has thus become an industry standard analytical tool to analyze charge heterogeneity and has been routinely used as a product release method.

Variant enrichment is also a valuable tool to understand the effects of charge sub-populations on drug efficacy, and can help to define product specification limits. However, most current enrichment methods typically rely on ion exchange chromatography, and the resulting fractions may not always correlate with release icIEF peak profiles.

Thus, there exists a need for methods for specifically and sensitively enriching charge variants of a therapeutic protein in a manner consistent with icIEF charge heterogeneity analysis.

This disclosure sets forth a novel method for enriching charge variants of a protein of interest. The method includes subjecting a sample including a protein of interest to icIEF analysis followed by collecting fractions of the focused sample. The icIEF analysis may be conducted either in normal polarity, with basic species mobilizing first out of the capillary, or in reverse polarity, with acidic species mobilizing first out of the capillary. The sample may be, for example, a drug substance. The sample may also be a partially enriched charge variant sample, for example a fraction collected from ion exchange chromatography separation of a drug substance. Collected fractions may be iteratively subjected to icIEF analysis for charge variant analysis and optimization of charge variant enrichment. Fractions enriched for a charge variant of interest may be collected and combined to yield an enriched charge variant sample. The enriched charge variant sample may then be subjected to additional characterization and analysis, for example to characterize drug potency of a charge variant of a therapeutic protein. Novel aspects of this disclosure include, for example, enrichment of charge variants fractionated with icIEF; sequential, back-to-back icIEF runs in reverse polarity; and the use of partially enriched charge variants as an icIEF starting material for further enrichment.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein of interest can be a recombinant protein, an antibody, a bi specific antibody, a multispecific antibody, antibody fragment, monoclonal antibody, fusion protein, scFv and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. In different embodiments of the invention, the FRs of the anti-big-ET-1 antibody (or antigen-binding portion thereof) may be identical to the human germline sequences or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Muller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Ser. No. 12/823,838, filed Jun. 25, 2010; U.S. Ser. No. 13/488,628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/223,430 filed Jul. 29, 2016; and U.S. Ser. No. 15/814,095, filed Nov. 15, 2017.

As used herein “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof.

In some exemplary embodiments, the protein of interest or a charge variant thereof can have a pI in the range of about 4.5 to about 9.0. In an exemplary embodiment, the pI of the protein of interest or a charge variant thereof is about 4.5, about 5.0, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, or about 9.0. In some exemplary embodiments, the types of protein of interest in the compositions can be more than one.

In some exemplary embodiments, the protein of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NSO, NS1 cells or derivatives thereof).

In some exemplary embodiments, the sample including the protein of interest can be prepared prior to or following icIEF analysis. Preparation steps can include alkylation, reduction, denaturation, and/or digestion.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N(Asp-N), endoproteinase Arg-C(Arg-C), endoproteinase Glu-C(Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

As used herein, the term “charge variant” or “variant” of a polypeptide refers to a polypeptide comprising an amino acid sequence that is at least about 70-99.9% (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.5, 99.9%) identical or similar to a referenced or native amino acid sequence of a protein of interest. A sequence comparison can be performed by, for example, a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment). Variants of a polypeptide may also refer to a polypeptide comprising a referenced amino acid sequence except for one or more (e.g., 1, 2, 3, 4, 5, 6, 7, 8, 9 or 10) mutations such as, for example, missense mutations (e.g., conservative substitutions), nonsense mutations, deletions, or insertions. The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.; the entire teachings of which are herein incorporated.

Some variants can be covalent modifications that polypeptides undergo, either during (co-translational modification) or after (post-translational modification or “PTM”) their ribosomal synthesis. PTMs are generally introduced by specific enzymes or enzyme pathways. Many occur at the site of a specific characteristic protein sequence (e.g., signature sequence) within the protein backbone. Several hundred PTMs have been recorded and these modifications invariably influence some aspect of a protein's structure or function (Walsh, G. “Proteins” (2014) second edition, published by Wiley and Sons, Ltd., ISBN: 9780470669853, the entire teachings of which are herein incorporated).

In certain exemplary embodiments, a protein composition can comprise more than one type of variant of a protein of interest. Such variants can include both acidic species and basic species. Acidic species are typically the variants that elute earlier than the main peak from CEX or later than the main peak from AEX, while basic species are the variants that elute later than the main peak from CEX or earlier than the main peak from AEX. In an exemplary embodiment, basic species may migrate earlier than the main peak from IEF and acidic species may migrate later than the main peak from IEF in normal polarity. In an exemplary embodiment, basic species may migrate later than the main peak from IEF and acidic species may migrate earlier than the main peak from IEF in reverse polarity.

As used herein, the terms “acidic species,” “AS,” “acidic region,” and “AR,” refer to the variants of a protein which are characterized by an overall acidic charge.

In certain embodiments, the sample can comprise more than one type of acidic species variant. For example, but not by way of limitation, the total acidic species can be categorized based on chromatographic retention time of the peaks appearing, or by UV or other absorbance peaks generated using IEF.

Among the chemical degradation pathways responsible for acidic or basic species, the two most commonly observed covalent modifications occurring in proteins and peptides are deamination and oxidation. Methionine, cysteine, histidine, tryptophan, and tyrosine are some of the amino acids that are most susceptible to oxidation: Met and Cys because of their sulfur atoms and His, Trp, and Tyr because of their aromatic rings.

As used herein, the terms “oxidative species,” “OS,” or “oxidation variant” refer to the variants of a protein formed by oxidation. Such oxidative species can also be detected by various methods, such as ion exchange, for example, WCX-10 HPLC (a weak cation exchange chromatography), or IEF. Oxidation variants can result from oxidation occurring at histidine, cysteine, methionine, tryptophan, phenylalanine and/or tyrosine residues.

As used herein, the terms “basic species,” “basic region,” and “BR,” refer to the variants of a protein, for example, an antibody or antigen-binding portion thereof, which are characterized by an overall basic charge, relative to the primary charge variant species present within the protein. For example, in recombinant protein preparations, such basic species can be detected by various methods, such as ion exchange, for example, WCX-10 HPLC (a weak cation exchange chromatography), or IEF. Exemplary variants can include, but are not limited to, lysine variants, isomerization of aspartic acid, succinimide formation at asparagine, methionine oxidation, amidation, incomplete disulfide bond formation, mutation from serine to arginine, aglycosylation, fragmentation and aggregation. Commonly, basic species elute later than the main peak during CEX or earlier than the main peak during AEX analysis. (Chromatographic analysis of the acidic and basic species of recombinant monoclonal antibodies. MAbs. 2012 Sep. 1; 4(5): 578-585. doi: 10.4161/mabs.21328, the entire teaching of which is herein incorporated by reference.)

In certain embodiments, the sample can comprise more than one type of basic species variant. For example, but not by way of limitation, the total basic species can be divided based on chromatographic retention time of the peaks appearing, or based on UV or other absorbance peaks generated using IEF. Another example in which the total basic species can be divided can be based on the type of variant—for example, structure variants or fragmentation variants.

As used herein, a “sample” can be obtained from any step of the bioprocess, such as cell culture fluid (CCF), harvested cell culture fluid (HCCF), any step in the downstream processing, drug substance (DS), or a drug product (DP) comprising the final formulated product. In some other specific exemplary embodiments, the sample can be selected from any step of the downstream process of clarification, chromatographic production, viral inactivation, or filtration. In some specific exemplary embodiments, the drug product can be selected from manufactured drug product in the clinic, shipping, storage, or handling. In some embodiments, a sample is a further processed form of any of the aforementioned examples of samples. In an exemplary embodiment, a sample is a partially enriched charge variant sample; for example, a fraction collected from an ion exchange chromatography analysis of a drug substance or other sample.

As used herein, “isoelectric focusing” or “IEF”, also known simply as electrofocusing, is a technique for separating charged molecules, usually proteins or peptides, on the basis of their isoelectric point (pI), for example, the pH at which the molecule has no charge. IEF works because, in an electric field, molecules in a pH gradient will migrate towards their pI. A variety of techniques for conducting IEF exist, all of which are encompassed in the term IEF as used herein where relevant. For example, in capillary isoelectric focusing (cIEF), samples travel through a capillary based on an applied electric field. A UV detector may be used at a point along the capillary to detect the time at which an analyte, such as a protein, traverses that point of the capillary. Because travel time through the capillary is directly related to the charge (pI) of the analyte, UV signal from a point in the capillary over time can be represented as a UV trace, which represents the varying charges (pI) of sample components. In an exemplary embodiment, a UV trace generated by cIEF represents charge variants of a protein of interest, with each UV peak representing a significant charge variant.

As used herein, the term “ampholyte” refers to a molecule that contains both acid and base functionality. Ampholytes can serve as pH buffers. Ampholytes, or “carrier ampholytes”, may be used to affect the mobility of analytes in capillary electrophoresis, for example in cIEF. Ampholytes may be aliphatic oligo-amino oligo-carboxylic acid molecules of varying length and branching. Mixtures of ampholytes may be selected according to their pI. Many ampholytes suitable for capillary electrophoresis are commercially available, for example Pharmalytes (Cytiva), Bio-Lytes (Bio-Rad), and AESlytes (Advanced Electrophoresis Solutions).

As used herein, the term “anolyte” refers to a low pH electrolytic solution mainly comprising anionic species. An anolyte is maintained in contact with an anode in cIEF. As used herein, the term “catholyte” refers to a high pH electrolytic solution mainly comprising cationic species. A catholyte is maintained in contact with a cathode in cIEF. A cIEF solution may further comprise an anodic stabilizer and a cathodic stabilizer.

Variations of cIEF may also be used, for example, imaged cIEF (icIEF). Examples of suitable devices for performing icIEF analysis include CElnfinite (Advanced Electrophoresis Solutions), iCE3 (ProteinSimple) and Maurice (ProteinSimple). Following focusing of charge variants in a sample, icIEF analysis may further comprise a mobilization step, wherein pressure is used to mobilize a focused sample past a detection window, and may additionally mobilize a focused sample out of the separation capillary. Fractions may be collected from the mobilized sample. Collected fractions may correspond to a charge variant of interest. A fraction may comprise more than one charge variant. A charge variant may be collected in more than one fraction.

cIEF analysis typically proceeds with “normal polarity”, wherein an anode is located towards the sample inlet and a cathode is located at the opposite end of the capillary, and basic species migrate first through the capillary towards the cathode. In an exemplary embodiment, cIEF analysis may proceed with “reverse polarity”, wherein a cathode is located towards the sample inlet and an anode is located at the opposite end of the capillary, and acidic species migrate first through the capillary towards the anode. Using reverse polarity may also include reversing the loading of anolytes and/or catholytes in the capillary, and modifying the mobilization flow rate in order to counteract the electroosmotic flow.

As used herein, the term “mass spectrometer” includes a device capable of identifying specific molecular species and measuring their accurate masses. The term is meant to include any molecular detector into which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSn, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMS SA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

In some exemplary embodiments, enrichment and/or characterization of a protein of interest and/or a charge variant of interest can be performed under native conditions. As used herein, the term “native conditions” can include performing mass spectrometry under conditions that preserve non-covalent interactions in an analyte. For detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE1176-1192 (2015).

It is understood that the present invention is not limited to any of the aforesaid protein(s), antibody(s), pI(s), protein alkylating agent(s), protein denaturing agent(s), protein reducing agent(s), digestive enzyme(s), hydrolyzing agent(s), charge variant(s), charge variant(s), post-translational modification(s), sample(s), IEF system(s), ampholyte(s), mass spectrometer(s), database(s), or bioinformatics tool(s), and any protein(s), antibody(s), pI(s), protein alkylating agent(s), protein denaturing agent(s), protein reducing agent(s), digestive enzyme(s), hydrolyzing agent(s), charge variant(s), charge variant(s), post-translational modification(s), sample(s), IEF system(s), ampholyte(s), mass spectrometer(s), database(s), or bioinformatics tool(s) can be selected by any suitable means.

The present invention will be more fully understood by reference to the following Examples. They should not, however, be construed as limiting the scope of the invention.

### EXAMPLES

### Example 1. Enrichment of Low Abundance Charge Variant Species from Drug Substance

A method for enriching charge variants of a protein of interest is disclosed herein. As proof of concept, the method of the present invention was used to enrich and characterize a low abundance charge variant species from drug substance.

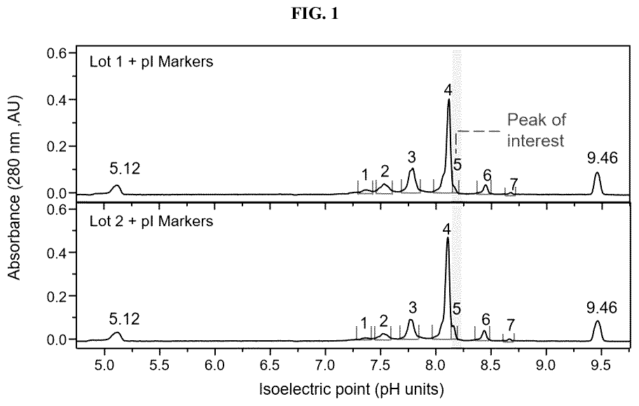

Two formulated drug substance (FDS) lots of a therapeutic protein, mAb1, were characterized using icIEF, as shown in Table 2. A minor difference was found in the relative abundance of a poorly-resolved, basic shoulder variant, peak 5. Electropherograms of lot 1 and lot 2 are shown in FIG. 1, demonstrating a poor separation of peaks 4 and 5.

TABLE 2
Charge variant peaks of mAb1 lot 1 and lot 2 by icIEF

| | Nominal Isoelectric | Peak Area % | | | |
|---|---|---|---|---|---|
| Peak | Point (pI) | Lot 1 | | Lot 2 | |
| 1 | 7.36 | 1.8 | (0.1) | 0.9 | (0.1) |
| 2 | 7.53 | 7.3 | (0.4) | 4.8 | (0.2) |
| 3 | 7.78 | 21.5 | (0.1) | 16.1 | (0.1) |
| 4 | 8.11 | 62.9 | (0.2) | 67.8 | (0.2) |
| 5 | 8.15 | 1.7 | (0.1) | 5.1 | (0.0) |
| 6 | 8.44 | 4.2 | (0.2) | 4.7 | (0.1) |
| 7 | 8.67 | 0.6 | (0.0) | 0.6 | (0.1) |

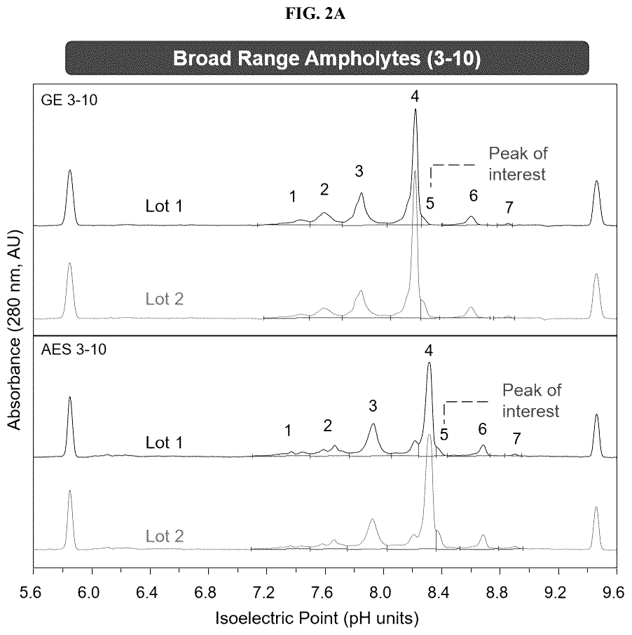

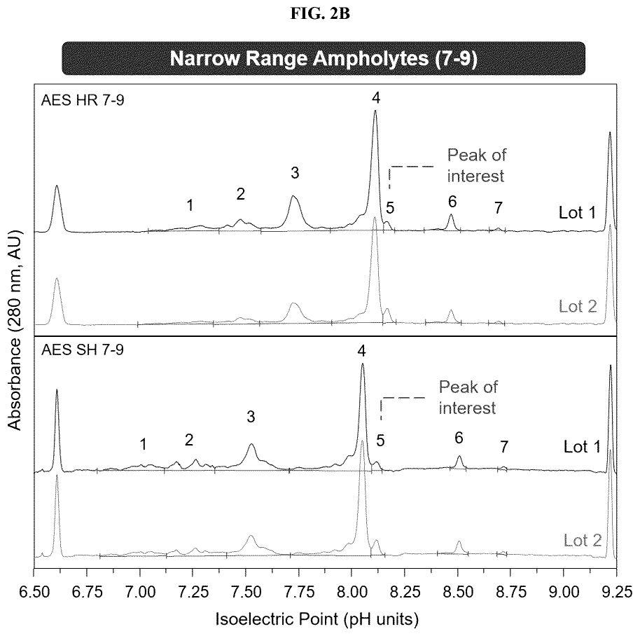

In order to improve the separation of mAb1 charge variants to allow for characterization of peak 5, several icIEF parameters were varied and compared, including, for example, sample load, polarity, carrier ampholytes, focusing time, mobilization time, peak collection intervals, and fractionation trigger absorbance. A comparison of separation profiles using varied ampholytes is shown in FIG. 2A and FIG. 2B. Ampholytes compared included Pharmalyte 3-10 (Cytiva), AESlyte 3-10, HR AESlyte 7-9, and SH AESlyte 7-9 (Advanced Electrophoresis Solutions).

The best resolution between peaks 4 and 5 was achieved using narrow range ampholytes between pH 7 and 9. In particular, super-high resolution AESlyte with pH range 7-9 (SH 7-9) ampholytes both resolved peaks 4 and 5 and additionally reduced contamination of peak 6 into peak 5, and thus yielded the optimal resolution. A comparison of peak 5 area quantitation in the two lots using different ampholytes is shown in Table 3. These results confirmed that lot 2 contained about 5% of peak 5, while lot 1 contained about 2.5%.

TABLE 3
Charge variant resolution using various icIEF ampholytes

| | Nominal Isoelectric | Peak 5 Area % | |
|---|---|---|---|
| Ampholyte | Point (pI) | Lot 1 | Lot 2 |
| GE 3-10 (reference data) | 8.15 | 1.7 | 5.1 |
| GE 3-10 | 8.26 | 2.4 | 5.2 |
| AES 3-10 | 8.36 | 2.1 | 5.4 |
| AES HR 7-9 | 8.17 | 2.2 | 4.7 |
| AES SH 7-9 | 8.12 | 2.5 | 4.9 |

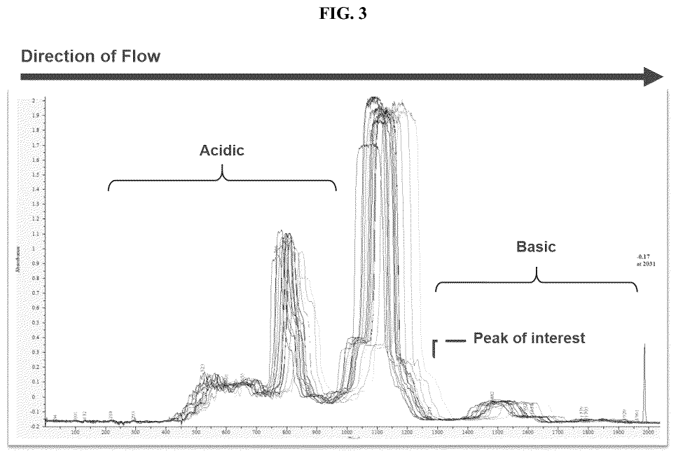

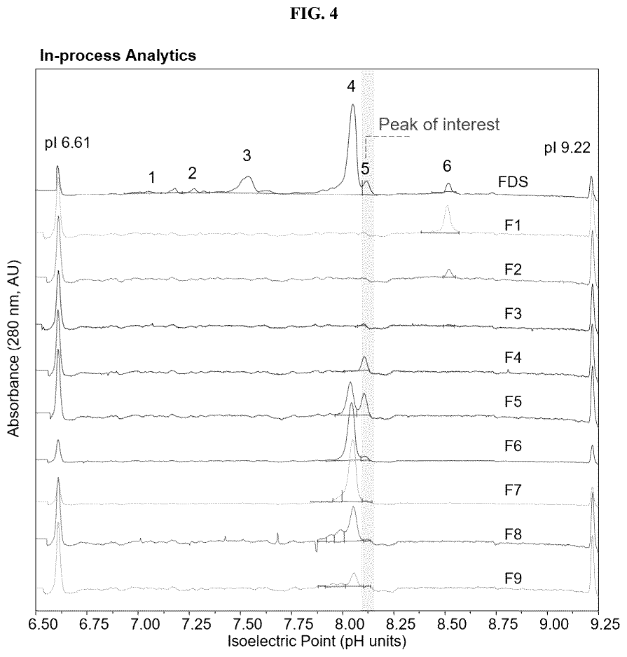

In order to determine whether the differing abundance of the basic charge variant peak 5 may have a clinical impact on mAb1, a method was developed for specific enrichment of peak 5 to allow for functional characterization. Lot 1 and lot 2 samples were subjected to icIEF analysis in 15 back-to-back injections using AESlytes SH 7-9, as shown in FIG. 3. The starting material for each injection was 16 μg of FDS. Following focusing of the samples, the focused samples were mobilized out of the capillary. Fractions were collected and subjected to icIEF analysis to characterize the charge variants in each fraction, as shown in FIG. 4. The peak of interest, peak 5, was enriched in fraction F4 (over 95% of peak 5) and fraction F5.

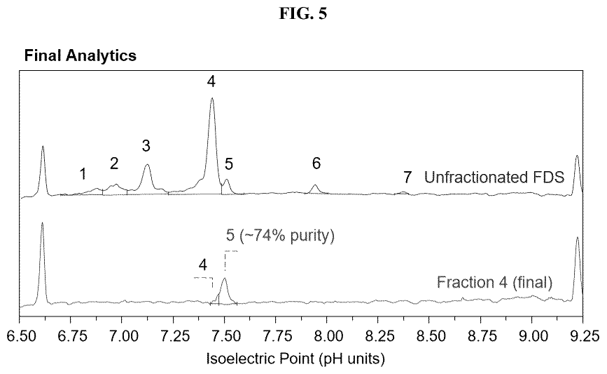

To ensure high purity, the fractionation scheme was further optimized, with about 20 injections performed per day for four days, for a total of 80 injections. Focusing time and mobilization time was minimized in order to maximize the number of overnight injections and increase the total amount of material collected. The final enriched fraction is shown in FIG. 5, with about 74% purity of the peak 5 charge variant.

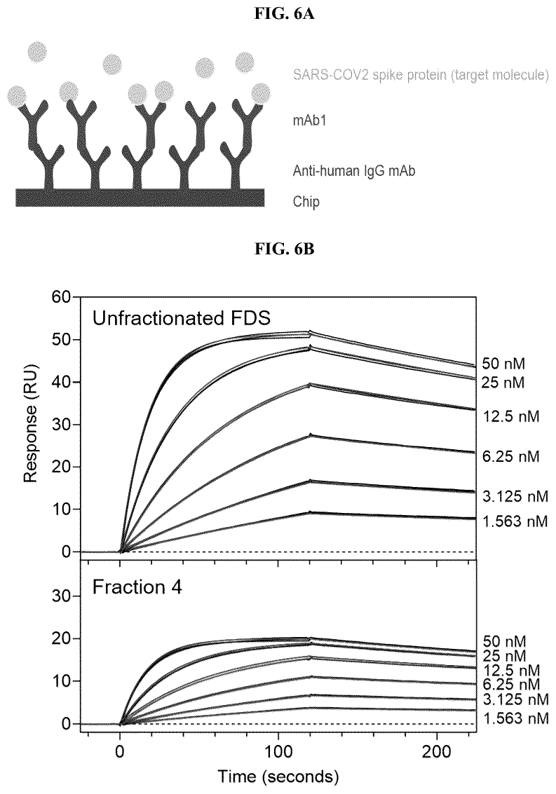

The method of charge variant enrichment using icIEF allowed for further characterization of the enriched charge variant species. A target-binding assay was used to assess the ability of the mAb1 charge variant to bind to its target, as illustrated in FIG. 6A. mAb1 is a human anti-SARS-CoV-2 antibody; an anti-human IgG mAb was used to capture mAb1 to a chip, and a ruthenium-labeled SARS-CoV-2 spike protein (the target molecule of mAb1) was used to label captured mAb1, providing a target-binding-sensitive measure of mAb1. Results are shown in Table 4 and FIG. 6B with varying concentrations of target molecule.

TABLE 4
Target binding of mAb1 charge variant

| | mAb | ka | kd | KD | Rmax | Rmax/mAb | t½ |
|---|---|---|---|---|---|---|---|
| mAb1 | Capture (RU) | (1/Ms) | (1/s) | (nM) | (RU) | Capture | (min) |
| Unfractionated FDS | 126 | 1.10E+06 | 1.57E−03 | 1.43 | 53 | 0.42 | 7 |
| Fraction 4 | 53 | 1.16E+06 | 1.62E−03 | 1.39 | 20 | 0.37 | 7 |

mAb1 capture level was reduced in the enriched charge variant sample, fraction 4, compared to the unfractionated FDS, potentially due to a lower protein concentration. Based on this analysis of the enriched charge variant species, the difference in relative abundance of the charge variant species between lot 1 and lot 2 as detected by icIEF likely does not pose a significant risk to drug quality. Using the method of the present invention, the particular charge variant detected by icIEF could be enriched and characterized to inform further drug development.

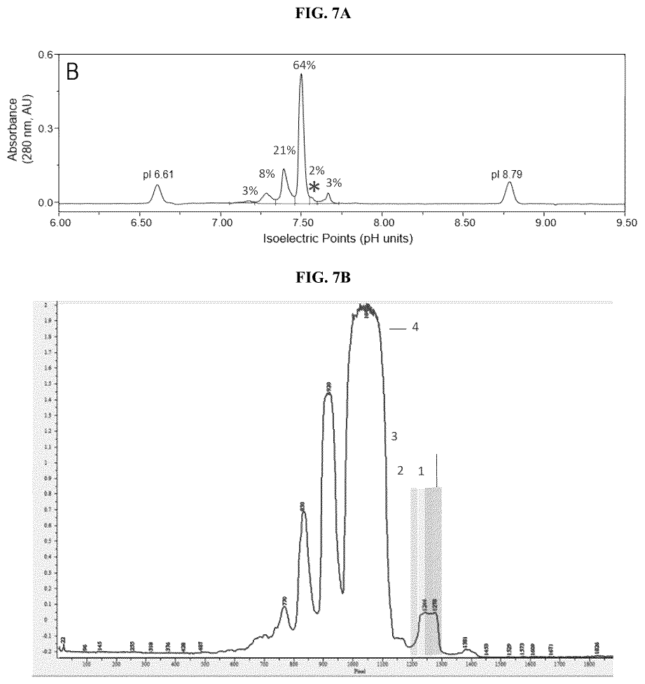

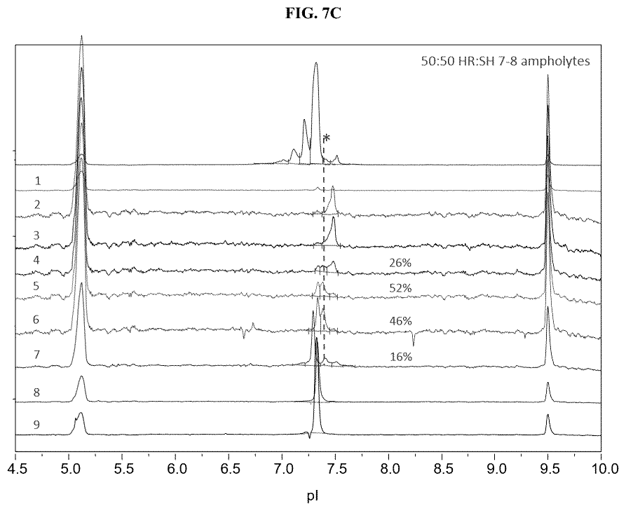

The charge variant enrichment method of the present invention was employed for another therapeutic antibody, mAb2. icIEF separation of mAb2 DS charge variants shows a small shoulder peak on the basic side of the main peak, as shown in FIG. 7A. In order to enrich this basic peak of interest, mAb2 DS was run on an icIEF capillary using 5-8 mg/mL protein load in 4 μL, 2 M urea, and 4% narrow range ampholytes with pI 6-8. Eight fractions were collected as shown in FIG. 7B and FIG. 7C, and the basic peak of interest was enriched to about 52% purity.

### Example 2. Reverse Polarity icIEF for Separation of Acidic Species

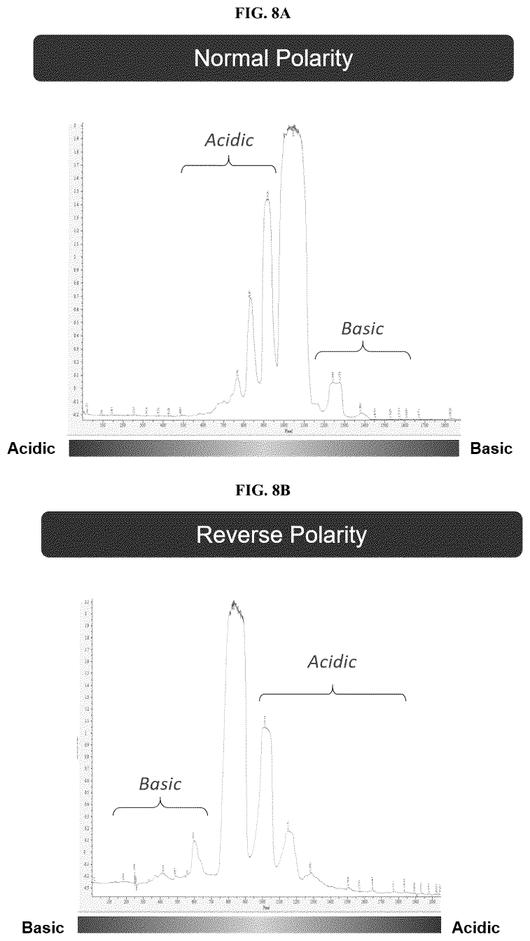

Normal polarity icIEF results in the migration of basic species followed by acidic species, as shown in FIG. 8A. Normal polarity is ideal for optimizing the separation of basic variants without contamination from the main peak. Acidic species, however, may be contaminated by the main peak, reducing the purity of any collected acidic charge variant species and hampering any analysis of enriched acidic charge variant species. Disclosed herein is the development of reverse polarity icIEF, wherein the positions of the cathode and anode are reversed, causing acidic species to migrate first, followed by basic species, as shown in FIG. 8B. The migration of acidic species first may reduce or eliminate contamination of acidic species by the main peak.

The benefits of reverse polarity icIEF as described above apply as well to any sample comprising multiple peaks, wherein a more abundant peak would migrate first if normal polarity were used, thus leading to carryover contamination in later peaks. For simplicity, descriptions of reverse polarity icIEF may refer to “acidic” peaks, but it should be understood that the acidity is only relative to other peaks in the sample, and not necessarily relative to a main peak or neutral pH. For example, a first basic peak may be contaminated due to a more abundant and more basic second basic peak, and therefore enrichment of the first basic peak (in the context of this sample, the relatively acidic peak) may be improved by using reverse polarity icIEF.

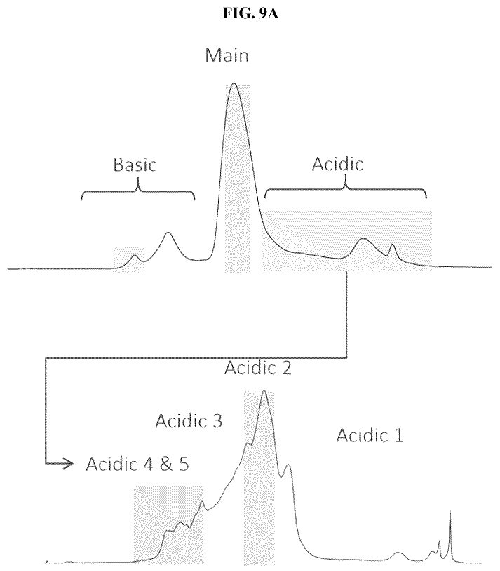

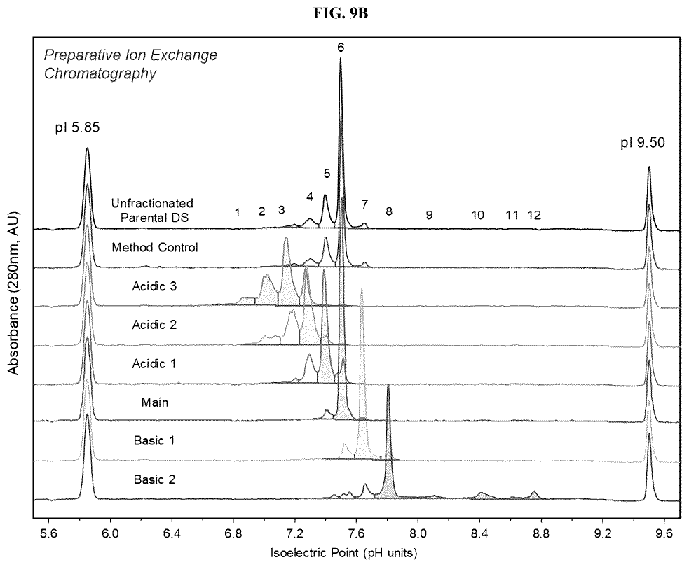

mAb2 DS lots are typically subjected to two enrichment steps: an anion exchange step, from which an acidic pool is collected for further processing, and a subsequent cation exchange step, as shown in FIG. 9A. However, even after two purification steps, acidic fractions may remain heterogeneous, as shown in FIG. 9B. This is of interest for mAb2 because acidic charge variants are known to exhibit reduced potency in a cell-based bioassay, particularly in acidic fraction 3, as shown in Table 5. Due to the heterogeneity of the acidic fractions, it is unknown if the reduced potency can be attributed to a specific acidic peak, and therefore a specific charge variant.

TABLE 5
Charge variant species of mAb2 by two-
step ion exchange chromatography

| Sample | Purity (%) | Potency (%) |
|---|---|---|
| Drug Substance | NA | 97.5 |
| Method Control | NA | 123 |
| Acidic 3 | 81.7 | 35.4 |
| Acidic 2 | 52.5 | 87.4 |
| Acidic 1 | 61.7 | 107 |
| Main | 92.3 | 97.8 |
| Basic 1 | 82.8 | 125 |
| Basic 2 | 81.1 | 135 |

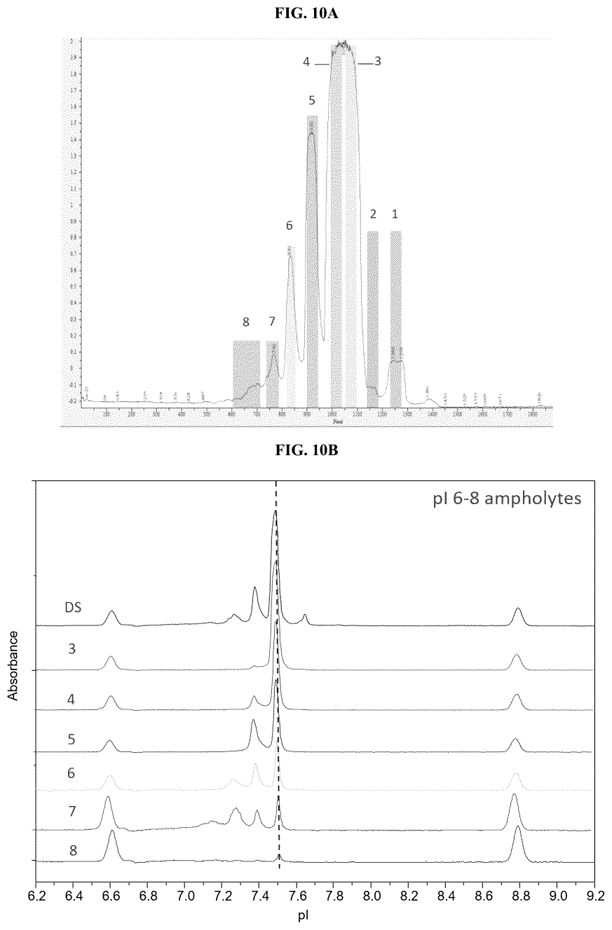

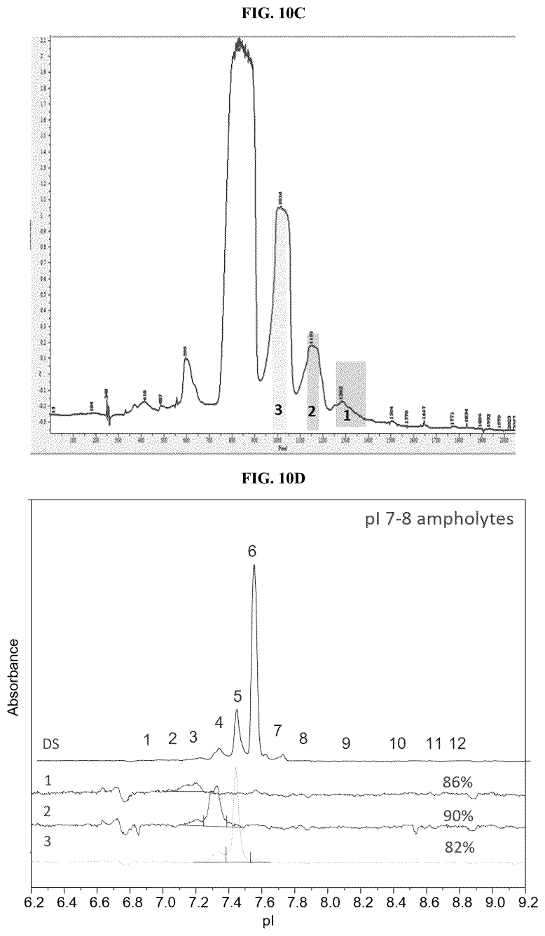

mAb2 drug substance was subjected to normal polarity icIEF, causing basic species to mobilize through the capillary first, as shown in FIG. 10A with the acidic peaks labeled 6, 7 and 8. Acidic fractions collected from this separation are contaminated with significant amounts of the main peak, as shown in FIG. 10B.

mAb2 drug substance was then subjected to reverse polarity icIEF, causing acidic species to mobilize through the capillary first, as shown in FIG. 10C with acidic peaks 1, 2 and 3 indicated. An optimization required for reverse polarity icIEF included a higher mobilization flow rate in order to counter-act the electroosmotic flow (EOF). The separated acidic peaks could then be mobilized out of the capillary and collected as fractions, with 86%, 90%, and 82% purity for acidic peaks 1, 2, and 3 respectively, as shown in FIG. 10D. Further analysis on enriched charge variants of interest could be conducted to determine which particular charge variants may be responsible for enhanced or reduced product quality.

### Example 3. Enrichment of Subvariants from a Partially Enriched Sample

As described above, a benefit of the method of the present invention is the ability to collect enriched charge variant fractions corresponding to the charge variants detected using icIEF, in contrast to the conventional method of detecting charge variants using icIEF and collecting fractions using ion exchange chromatography, which may not directly correspond to each other. An additional benefit of the method of the present invention is the ability to further enrich partially enriched charge variant samples, for example partially enriched charge variant fractions collected using ion exchange chromatography. Charge variant species that co-segregate in a single fraction from ion exchange chromatography may be separated and enriched in separate fractions using the icIEF method of the present invention.

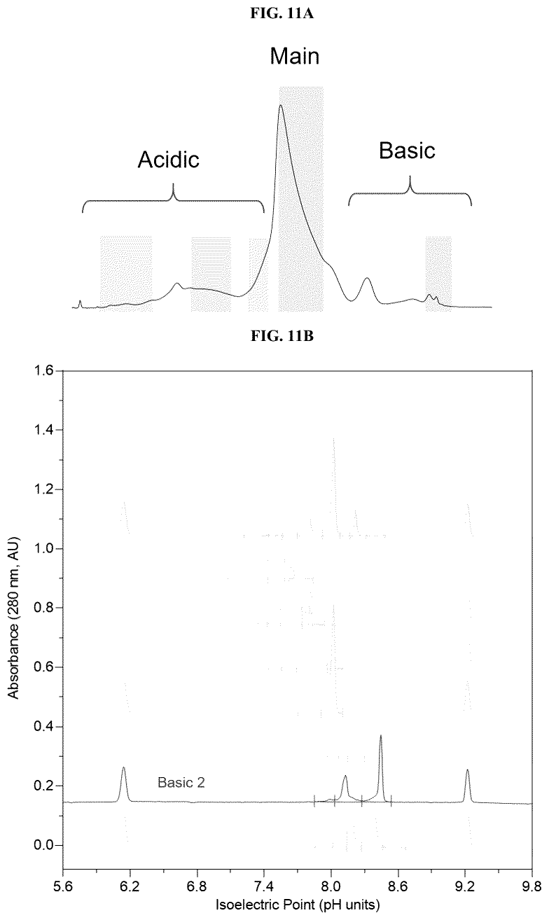

mAb3 is a therapeutic antibody whose charge variants have previously been enriched from DS using strong cation exchange chromatography (SCX), yielding three acidic, one main, and three basic fractions, as shown in FIG. 11A. The second basic fraction, Basic 2, is heterogeneous, with distinct peaks that were not individually enriched using SCX, as shown in FIG. 11B. Therefore, a charge variant enrichment method using icIEF was optimized in order to enrich the distinct charge variant species of Basic 2.

Because it comprises a partially enriched sample of a therapeutic drug substance, Basic 2 represents a limited sample. In order to conserve material during method optimization, a pI marker mixture was used as a proxy for the species in Basic 2. The pI values of ProteinSimple reagents were compared to the pI of Basic 2 species in order to select suitable proxy markers, as shown in Table 6.

TABLE 6
Comparison of ProteinSimple pI markers
to pI of mAb3 basic species

| Reagent Number | pI Value | Basic 2 Species |
|---|---|---|
| 102221 | 2.85 | — |
| 102018 | 3.21 | — |
| 102222 | 3.59 | — |
| 102350 | 4.22 | — |
| 102223 | 4.65 | — |
| 102224 | 5.12 | — |
| 102225 | 5.85 | — |
| 102220 | 6.14 | — |
| 102409 | 6.61 | — |
| 102226 | 7.05 | — |
| 102227 | 7.40 | — |
| 102170 | 7.55 | — |
| 102407 | 7.65 | — |
| 102171 | 7.90 | 7.99 |
| 102408 | 8.18 (+0.28) | 8.12 (+0.13) |
| 102229 | 8.40 (+0.22) | 8.44 (+0.32) |
| 102230 | 8.79 | — |
| 102231 | 9.22 | — |
| 102349 | 9.46 | — |
| 101996 | 9.5 | — |
| 102219 | 9.77 | — |
| 102232 | 10.1 | — |
| 102233 | 10.45 | — |

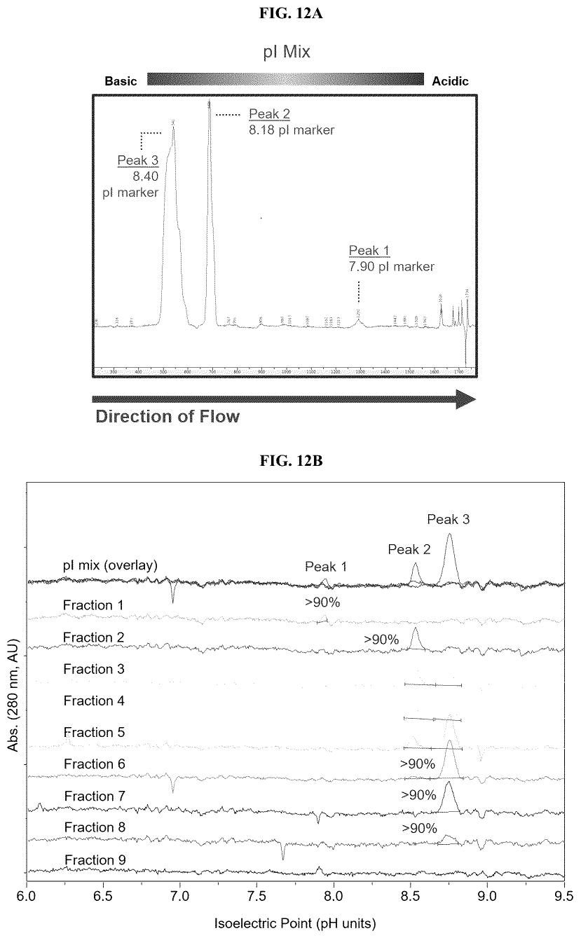

The proxy pI markers were combined in the same ratio of abundance as the Basic 2 peaks. In order to minimize carryover contamination of the most-abundant peak into earlier fractions, reverse polarity icIEF was used. Enrichment of proxy pI markers using reverse polarity icIEF and fraction collection is shown in FIG. 12A and FIG. 12B. Focusing time and mobilization time were minimized in order to maximize the number of overnight injections, increasing the total amount of material collected in order to optimize final purity. Each of the three peaks was collected with greater than 90% purity.

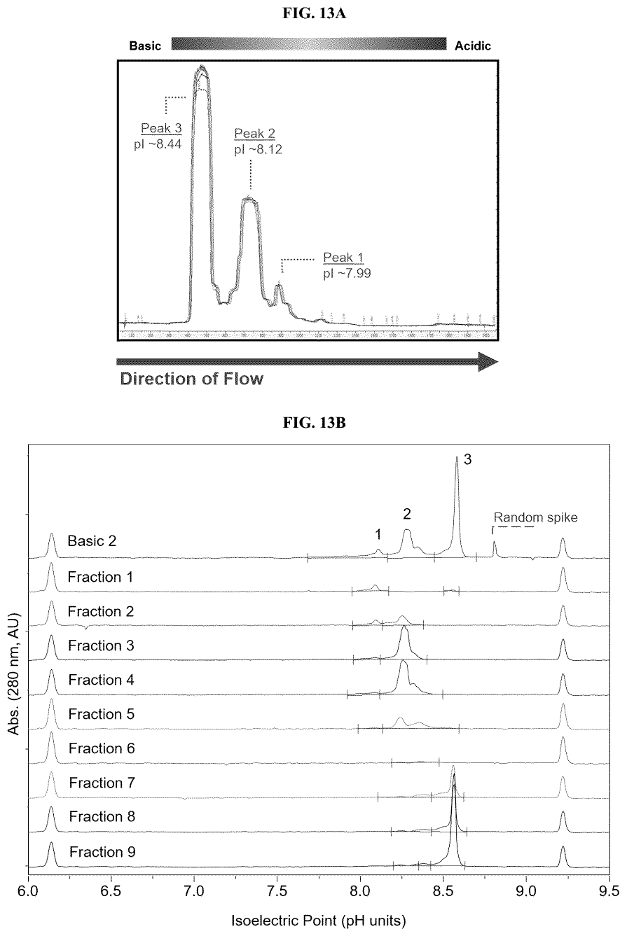

Basic 2 sample was subjected to the optimized reverse polarity icIEF method as shown in FIG. 13A and FIG. 13B. Fractions 2 through 5 were collected to form an enriched sample of the Basic 2 second peak, with greater than 90% purity. Fractions 7 through 9 were collected to form an enriched sample of the Basic 2 third peak, with greater than 90% purity. Thus, the method of the present invention was capable of using partially enriched samples from ion exchange chromatography analysis to further produce highly enriched samples of individual charge variant species.

In some exemplary embodiments, the method of the present invention may be applied for investigating unique and/or novel peaks for identity and potency characterization. In some other embodiments, the method of the present invention may be applied for secondary polishing for improved icIEF purity of intermediate acidic fractions. In other embodiments, the method of the present invention may be applied for better understanding post-translational modifications with differences in surface charge versus intrinsic or formal charge.

What is claimed is:

1. A method for enriching a charge variant of a protein of interest, comprising:
(a) subjecting a sample including a protein of interest to reverse polarity capillary isoelectric focusing to separate said charge variant of said protein of interest;
(b) collecting at least one fraction including said charge variant from said capillary isoelectric focusing step;
(c) repeating steps (a) and (b) at least once; and
(d) combining the fractions of steps (a) through (c) to produce a sample enriched for said charge variant of said protein of interest.

2. A method for enriching a charge variant of interest, comprising:
(a) subjecting a sample including a charge variant of interest and at least two other charge variants to ion exchange chromatography to separate said charge variants;
(b) collecting at least one fraction including said charge variant of interest and at least one other charge variant;
(c) subjecting said at least one fraction to capillary isoelectric focusing to separate said charge variants; and
(d) collecting at least one fraction including said charge variant of interest.
